(19) United States
(12) Patent Application Publication
(10) Pub. No.: US 2001/0055769 A1
(43) Pub. Date: Dec. 27, 2001

(54) Method of determining biological/molecular age

(76) Inventor: Michael D. Seidman, West Bloomfield, MI (US)

Correspondence Address: Allen M. Krass, GIFFORD, KRASS, GROH, SPRINKLE,, ANDERSON & CITKOWSKI, P.C., 280 N. Old Woodward, Suite 400, Birmingham, MI, 48009, US

(21) Appl. No.: 09/885,732

(22) Filed: Jun. 20, 2001

Related U.S. Application Data
(60) Provisional application No. 60/212,747, filed on Jun. 20, 2000.

(51) Int. Cl. C12Q 1/68; C12P 19/34

(52) U.S. Cl. 435/6; 435/91.1

(57) ABSTRACT

The molecular biological age of an individual, as opposed to the chronological age, is determined by extracting mitochondrial DNA from a physical specimen from the individual, performing molecular biological testing to detect aging deletions, quantifying the deletions and comparing the quantification with normative data for the quantification derived from a plurality of age groups of a population.

### RELATED APPLICATIONS

This application claims priority of U.S. Provisional Patent Application 60/212,747 filed Jun. 20, 2000 and is incorporated herein by reference.

### FIELD OF THE INVENTION

This invention relates to a method of determining the biological/molecular age of a human and more particularly to such a method which involves detection of and quantification of aging deletions and comparison of the quantified deletions with known normative data.

### BACKGROUND OF THE INVENTION

Aging is a complex process that involves metabolic and physiologic changes that lead to an increasing susceptibility to disease and ultimately death. In order to address the basis for this patent, an explanation of several of the major scientific hypotheses explaining aging will be discussed. There are many theories to explain the aging process. However, three leading theories have the greatest scientific support and include: the membrane hypothesis of aging (MHA), the telomerase theory of aging and the dysdifferentiation hypothesis of aging.

The membrane hypothesis of aging (MHA), also called the mitochondrial clock theory of aging, is based upon the progressive accumulation of oxidative damage and is directly related to this patent. This progressive damage occurs secondary to the action of reactive oxygen species (ROS) also known as free radicals which are generated in increasing quantities with age.^{39,40} ROS are known to damage DNA in general and mitochondrial DNA in specific, as well as cells and tissue. The mitochondrial DNA damage leads to reduced capacity for energy generation within the mitochondria and ultimately causes aging and death. This is the premise for the use of powerful antioxidants to perhaps slow the processes of aging.

The mitochondrion is a tiny structure inside a cell and is the primary generator of energy, in the form of adenosine tri-phosphate (ATP). Mitochondria have their own DNA which determines all of their functions. The mitochondrial DNA (MtDNA) is made up of 16569 base pairs that, when completely intact, makes energy for the body. However, subtle changes in the MtDNA have dramatic effects on mitochondrial function and energy production. Research in our laboratory, and in several others around the world, has identified a specific deletion (or elimination) in mitochondrial DNA that is known to occur in response to aging. It is called the common aging deletion and consists of 4977 base pairs. It is not difficult to comprehend that if you remove approximately ⅓ of the mitochondrial DNA you will have significant problems with energy generation. It has been found that even minor amounts of this deletion severely alter energy production and cellular function. This deletion can be measured in our body and can effectively be used as a molecular test for aging, and although not yet commercially available, our laboratory has been studying this deletion in mice, rats and humans for the past 6 years. We also know that the common aging deletion can be identified in cells as early as 30 years of age, and there are some medical conditions where it can be seen even at 1 year of age. There are other mtDNA deletions that can occur in response to aging, such as the 520 bp deletion, etc.

Studies from our laboratory have demonstrated an age-dependent increase in the presence of the common mitochondrial deletion (MtDNA⁴⁹⁷⁷ in human; MtDNA⁴⁸³⁴ in rat) (Seidman et al). Specifically, we identified the common aging deletion in one of fifteen young rats, while eleven of fourteen aged rats had the MtDNA del. The aged rats also had hearing loss, and even more interesting is that the three aged rats without the deletion had better hearing when compared to the eleven with the deletion. Additionally, we were able to study mitochondrial function in aged rats and humans and as you would predict, it is significantly reduced compared to the young subjects. (Seidman et al. in progress). Human studies have revealed the presence of this MtDNA del in white blood cells of patients with age-related hearing loss more often than in control patients (Ueda et al. 1998). Two other human studies have identified the common aging deletion (MtDNA⁴⁹⁷⁷) in patients with age-related hearing loss more than in control subjects (Bai et al. 1997; Fischel-Ghodsian et al. 1998).

### SUMMARY OF THE INVENTION

We propose to use this sensitive molecular biologic test to study MtDNA dels and determine, with accuracy, an individual's “molecular age”. Preliminary evidence and logic predicts that even though two people may have the same chronologic age, that due to variations in lifestyle, diet, socio-economic factors, and genetics their molecular age may well be very different. For example: There are two forty year old men: One lives in Northern Michigan (at sea level) has an excellent diet, exercises regularly, supplements with specific nutrients, doesn't smoke or spend much time in the sun. Additionally, this Michigan native has a body mass index of 22 (normal =<25). Contrast this to another 40 year old man who lives in Colorado (about 5000 feet above sea level, this provides for more ionizing radiation), has a poor diet, rarely exercises, doesn't use nutritional supplements, smokes ½ pack of cigarettes per day and is always out in the sun. Additionally, his BMI is 32 (considered obese). Even though both are 40 years old, analysis of their mitochondria shows vast differences with 10-200 fold increases in the mtDNA deletion in the gentleman from Colorado. In essence, the man from Colorado has more rapid aging and in reality has the mitochondria of a 65 year old. This information is a wake up call to alter one's lifestyle immediately. This test has significant commercial value and provides critical information regarding one's molecular age and an indirect measure of long-term ROS damage.

It is known that certain tissues are more susceptible to oxidative damage (damage from free radicals) and reduced energy supply. This is particularly true for tissues that no longer make new cells. For example, brain, eye, inner ear, and all muscle can accumulate high amounts of these deletions and they become more susceptible to free radical damage than other tissues. Thus, increased oxidative damage that is associated with aging preferentially affects these tissues.

There are two other leading theories of aging: 1. The Telomerase theory of aging and 2. The Dysdifferentiation theory of aging. The end of a chromosome is made up of a structure called the Telosome. The tip of the Telosome is a region of repeating DNA sequences and proteins called the Telomere. The telomerase theory of aging suggests that there is a reduction in telomere length over time. (Pommier J P. Lebeau J. Ducray C. et al. Chromosomal instability and alteration of telomere repeat sequences. Biochimie. 77(10):817-25, 1995). Another way to look at this is to consider the telosome as similar to the tail of a rattlesnake. There are a finite number of rings on a telosome (or a rattlesnake) and the theory suggests that each time the telosome reproduces one ring is lost. When there are only a few rings of the telosome left, death is eminent. Interestingly, the activation of the enzyme responsible for making these rings disappear (telomerase enzyme) can be manipulated experimentally. However, it has already been found that cancer alters the telomerase enzyme, thereby becoming immortal. It is felt that special genes, called viral oncogenes, may produce immortality of a cell or tissue by activating telomerase, thus effectively preventing telomere shortening and sustaining cellular growth of tumors (Shay J W. Wright W E. Telomerase activity in human cancer. Current opinion in oncology. 8(1):66-71, 1996 January). Although many aspects of telomerase activity remain undefined, it has been hypothesized that the balance between telomere shortening and telomerase activity may underlie cellular aging processes. Furthermore, caution must be exercised when these genes are manipulated, because of the potential to trigger cancerous change.

The dysdifferentiation hypothesis suggests that there is a preprogrammed activation of genes that are deleterious to the cell and lead to activation of enzymes and reactions that are responsible for age-related changes. This line of reasoning was, in part, brought to the forefront from work elaborating control mechanisms of aging in the earthworm. Two main genes, Bax and BCl₂, have essential roles in cellular aging and immortality respectively. Scientists were able to increase the lifespan of the common earthworm by 30-40% by increasing the activity of the BCl₂ gene. However, once again, it has been shown that several cancers become immortal precisely by up-regulating the BCl₂ gene.

The process of aging is associated with many molecular, biochemical and physiological changes including increases in DNA damage, reduction in mitochondrial function, decreases in cellular water concentrations, ionic changes, and decreased elasticity of cellular membranes. One contributing factor to this process is altered vascular characteristics, such as reduced flow and vascular plasticity as well as increased vascular permeability (Prazma et al. 1990; Seidman et al. 1996). Atherosclerosis and high lipids and cholesterol further affect these situations and reduce the overall blood flow to many tissues in the body. These age-related changes result in reductions in oxygen and nutrient delivery and in waste elimination. (Gacek and Schuknecht 1969; Harkins 1981; Rosenhall et al. 1986; Hoeffding and Feldman 1987) These physiologic inefficiencies favor the production of ROS. Furthermore, there is support in the literature for age-associated reduction in enzymes that protect from ROM damage including superoxide dismutase, catalase and glutathione (Semsei et al. 1982; 1989; Richardson et al. 1987). Collectively, these changes enhance the generation of ROS.

### DETAILED DESCRIPTION OF A PREFERRED EMBODIMENT

### Protocol

Detection and Quantification of mtDNA Deletion (mtDNA⁴⁹⁷⁷)

(N-W Soong and N. Arnheim, Meth Enzymol., 421-431, 1996)

Primers (Designed in our Laboratory)

Mt1C: AGG CGC TAT CAC CAC TCT TGT TCG (13,176-13198)

Mt2: AAC CTG TGA GGA AAG GTA TTC CTG C (13,501-13,477)

Mt1A: GAA TTC CCC TAA AAA TCT TTG AAA T (8224-8247)

Primers are end-labeled with (K-³²P) ATP using T4 Polynucleotide Kinase. Unincorporated nucleotides are removed by spinning through P4 columns. These primer lots are prepared to give approximately 10×concentration for PCR (5 TM) and are diluted directly into the PCR mix.

### PCR Analysis

PCR is carried out in 50 Φ1 volumes in 1×PCR buffer, containing 1.5 mM MgCl₂

³²P end-labeled primer concentration is 0.5 ΦM.

Deoxy-nucleoside triphosphate (dNTPs) 200 ΦM.

2.5 Units of Taq polymerase.

100-1000 ng of genomic DNA.

Primers for Total mtDNA: Mt1C and Mt2, fragment size 324 bp.

Primers for Deletion: Mt1A and Mt2, fragment size 303 bp.

### Cycle Parameters

Initial denaturation at 94° for 3 min.

Denaturation at 94° for 30 sec. - - - \

Annealing at 54° for 30 sec 30 cycles

Extension at 72° for 1 min. - - - /

Followed by 7 min extension at 72°.

PCR conditions are identical for total and deletion-specific reactions except that deletion-specific reactions are run for 30 cycles and control PCR is carried out for 15 cycles,

### Polyacrylamide Gel Electrophoresis

After PCR, 10% (5 Φ1) of each reaction is electrophoresed through 8% polyacrylamide gel. The gel is dried and counts from each specific band are quantitated with a PhosphorImager (Biorad) after 15-24 hr exposure.

### Preparation of External Standards

For the construction of standard curves for deletion and control PCR, the respective PCR products are purified as a source of templates for the amplification reactions. Genomic DNA from aged heart tissues is used as a template for these preparative PCRs. The product bands are excised, electroeluted and concentrated by centrifuging through Centricon-10. These are aliquoted and stored at −20°. In order to develop “normal ranges” an acceptable standard is created by studying as few as 10 people and as many as 10,000 people in each decade ranging from 0-10, 11-20, 21-30, 31-40, 41-50, 51-60, 61-70, 71-80, 81-90, 91-100, 101-110, 111-120 years etc. Individual patients are then compared to a “normal” range thus providing a range for an acceptable amount of MtDNA deletion.

Serial dilutions of external standards were made and the range of dilutions over which the amplifications were exponential was determined. The plot of log counts versus log dilutions provides a good linear fit with a slope close to 1.

### Quantitation of Samples

Preliminary deletion and control PCR with unlabeled primers are performed on dilutions of DNA samples. The products signals are visually compared in ethidium bromide stained gels along with those of generated by amplification of the most concentrated standard dilution in the exponential range. The samples can then be diluted not to exceed the exponential range of the standard.

The PCR is then repeated with ³²P labeled primers. Both control and deletion standards are amplified in parallel with the samples. The products are quantified and the signal generated by each sample is then extrapolated from the appropriate standard curve to obtain the equivalent dilution of the standard stock that would have given the same signal. The percentage of the ratio of the deletion dilution to that of control dilution would then give the % ratio of mtDNA del to total mtDNA.

### Mitochondrial DNA Deletion Analysis By Serial Dilution

(N. S. Hamblet and F. J. Castora. Biochem Biophys Res Commun., 207:839-847, 1995)

Primers: Same as above.

PCR Reaction and Cycle Parameters: Same as above.

### Protocol

Total DNA was diluted in two ranges: one for deleted mtDNA amplification (250,000-976 pg) and one for wild type mtDNA amplification (500-1.95 pg). Samples were linearized with Bam H1 before amplification. PCR products were electrophoresed on a 8% polyacrylamide gel and visualized by ethidium bromide staining. Photographs were taken and negatives were scanned using a laser densitometer. The ratio of deleted to wild type mtDNA was determined by densitometric measurement of the intensity of each band and subsequent plotting of the optical density (OD) versus the log of weight of DNA in the reaction mix. The OD was adjusted so that the area of each DNA band was normalized by the size of the DNA fragment. The plots of deleted and wild type PCR products were examined to determine the logarithmic values on the x axis at which ODs of the deleted and undeleted PCR products were equivalent. The selected OD should be within the linear range of the density curve and has low standard deviation.

Having thus described my invention, I claim:

1. A method of determining the molecular age of an individual, comprising:
obtaining a physical specimen from the individual containing DNA;
extracting mitochondrial DNA from the sample;
performing molecular biological testing to analyze aging deletions in the mitochondrial DNA; and
comparing the quantity of deletion to predetermined normative data representing the quantity of deletions in the various age groups of a population to determine the molecular age of the individual.

2. The method of determining the molecular age of an individual of claim 1 wherein the step of extracting mitochondrial DNA from a specimen involves PCR amplification.

3. The method of determining the molecular age of an individual of claim 1 wherein the step of performing molecular biological testing to determine aging deletions involves performing gel electrophoresis and analyzing the number of rings in the telosomes.

4. The method of determining the molecular age of an individual of claim 1 wherein the step of performing molecular biological testing to determine aging deletions involves analysis of deleted and wild type PCR products to determine their ratio.

5. The method of determining the molecular age of an individual of claim 1 wherein the step of quantifying the deletion involves generating a quantity of mitochondrial DNA deletions.
